SEQUENCE LISTING (9 sequences)

SEQ ID NO: 1 - protein, 574 aa, Trametes cingulata
  SIGNAL (1)..(18)
MRFTLLTSLLGLALGAFAQSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 2 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 3 - protein, 957 aa, Bacillus sp.
  SIGNAL (1)..(29)
MAKKLIYVCLSVCLVLTWAFNVKGQSAHADGNTTTIIVHYFRPAGDYQPWSLWMWPKDGGGAEYDFNQPADSFGAVASADIPGNPSQVGIIVRTQDWTKDVSADRYIDLSKGNEVWLVEGNSQIFYNEKDAEDAAKPAVSNAYLDASNQVLVKLSQPLTLGEGASGFTVHDDTANKDIPVTSVKDASLGQDVTAVLAGTFQHIFGGSDWAPDNHSTLLKKVTNNLYQFSGDLPEGNYQYKVALNDSWNNPSYPSDNINLTVPAGGAHVTFSYIPSTHAVYDTINNPNADLQVESGVKTDLVTVTLGEDPDVSHTLSIQTDGYQAKQVIPRNVLNSSQYYYSGDDLGNTYTQKATTFKVWAPTSTQVNVLLYDSATGSVTKIVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDNVKTGIDSLKQLGITHVQLMPVFASNSVDETDPTQDNWGYDPRNYDVPEGQYATNANGNARIKEFKEMVLSLHREHIGVNMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPDDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDAVNEFDWSRKAQYPDVFNYYSGLIHLRLDHPAFRMTTANEINSHLQFLNSPENTVAYELTDHVNKDKWGNIIVVYNPNKTVATINLPSGKWAINATSGKVGESTLGQAEGSVQVPGISMMILHQEVSPDHGKK

SEQ ID NO: 4 - protein, 515 aa, Bacillus stearothermophilus
  mat_peptide (1)..(515)
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 5 - protein, 640 aa, Aspergillus niger
  SIGNAL (1)..(18)
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 6 - DNA, 1068 bp, Thermoascus aurantiacus
  CDS (1)..(1065)
  misc_signal (1)..(57)
  misc_feature (58)..(534)
  mat_peptide (535)..(1068)
atgcggctcgttgcttccctaacggccttggtggccttgtccgtacctgtctttcccgctgctgtcaacgtgaagcgtgcttcgtcctacctggagatcactctgagccaggtcagcaacactctgatcaaggccgtggtccagaacactggtagcgacgagttgtccttcgttcacctgaacttcttcaaggaccccgctcctgtcaaaaaggtatcggtctatcgcgatgggtctgaagtgcagttcgagggcattttgagccgctacaaatcgactggcctctctcgtgacgcctttacttatctggctcccggagagtccgtcgaggacgtttttgatattgcttcgacttacgatctgaccagcggcggccctgtaactatccgtactgagggagttgttccctacgccacggctaacagcactgatattgccggctacatctcatactcgtctaatgtgttgaccattgatgtcgatggcgccgctgctgccactgtctccaaggcaatcactcctttggaccgccgcactaggatcagttcctgctccggcagcagacagagcgctcttactacggctctcagaaacgctgcttctcttgccaacgcagctgccgacgcggctcagtctggatcagcttcaaagttcagcgagtacttcaagactacttctagctctacccgccagaccgtggctgcgcgtcttcgggctgttgcgcgggaggcatcttcgtcttcttcgggagccaccacgtactactgcgacgatccctacggctactgttcctccaacgtcctggcttacaccctgccttcatacaacataatcgccaactgtgacattttctatacttacctgccggctctgaccagtacctgtcacgctcaggatcaagcgaccactgcccttcacgagttcacccatgcgcctggcgtctacagccctggcacggacgacctggcgtatggctaccaggctgcgatgggtctcagcagcagccaggctgtcatgaacgctgacacctacgctctctatgcgaatgccatataccttggttgctaa

SEQ ID NO: 7 - protein, 355 aa, Thermoascus aurantiacus
MRLVASLTALVALSVPVFPAAVNVKRASSYLEITLSQVSNTLIKAVVQNTGSDELSFVHLNFFKDPAPVKKVSVYRDGSEVQFEGILSRYKSTGLSRDAFTYLAPGESVEDVFDIASTYDLTSGGPVTIRTEGVVPYATANSTDIAGYISYSSNVLTIDVDGAAAATVSKAITPLDRRTRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 8 - protein, 412 aa, Pyrococcus furiosus
  mat_peptide (1)..(412): Pyrococcus furiosus protease (Pfu)
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 9 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAVFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

(19) United States
(12) Patent Application Publication
(10) Pub. No.: US 2022/0186266 A1
(43) Pub. Date: Jun. 16, 2022

(54) Process For Producing A Fermentation Product

(71) Applicant: Novozymes A/S, Bagsvaerd (DK)

(72) Inventor: Nathaniel Edward KREEL, Louisburg, NC (US)

(73) Assignee: Novozymes A/S, Bagsvaerd (DK)

(21) Appl. No.: 17/600,767

(22) PCT Filed: Apr. 2, 2020
(86) PCT No.: PCT/US2020/026296

§ 371 Date: Oct. 1, 2021

Related U.S. Application Data
(60) Provisional application No. 62/828,268, filed on Apr. 2, 2019.

(51) Int. Cl. C12P 7/14; C12P 19/14; C12N 9/26; C12N 9/34; C12N 9/42; C12N 9/44; C12P 19/04; C12P 19/02; C12P 19/12

(52) U.S. Cl. CPC C12P 7/14; C12P 19/14; C12N 9/2414; C12Y 302/01001; C12N 9/2428; C12Y 302/01003; C12P 19/12; C12Y 302/01006; C12N 9/2457; C12Y 302/01041; C12Y 302/01015; C12P 19/04; C12P 19/02; C12N 9/244

(57) ABSTRACT

The present invention relates to processes of producing a fermentation product from starch containing material comprising (a) forming a slurry comprising the starch-containing material and water; (b) converting the starch-containing material into dextrins with an alpha-amylase; (c) saccharifying the dextrins using a carbohydrate source generating enzyme to form sugars; (d) fermenting sugars using a fermenting organism; (e) recovering the fermentation product to form whole stillage; (f) separating the whole stillage into a liquid fraction thin stillage and solid fraction wet cake; (g) hydrolyzing the thin stillage; (h) recycle a portion of the hydrolyzed thin stillage to steps (a); wherein the thin stillage in step (g) is hydrolyzed using a glucoamylase and/or polygalacturonase.

### REFERENCE TO A SEQUENCE LISTING

This application contains a Sequence Listing in computer readable form, which is incorporated herein by reference.

### FIELD OF THE INVENTION

The present invention relates a process of producing fermentation products, such as especially ethanol, from starch-containing material, wherein hydrolysed thin stillage (i.e., backset) at the backend of the process is recycled to the slurry tank at the frontend of the process.

### BACKGROUND OF THE INVENTION

At the backend of dry-grind ethanol plants (after distillation) whole stillage, which is rich in fiber, oil, protein, residual and unfermented sugars, and yeast cells, is fractionated (typically using a decanter centrifuge) into thin stillage (liquid fraction) and wet cake (solid fraction). The thin stillage is either partitioned to a series of evaporators to produce syrup or flows as backset back to the frontend of the plant (slurry tank) to be combined with fresh ground starch-containing material, e.g., corn or wheat, and fresh water in the formulation of the slurry.

Ethanol plants (see, e.g., FIG. 1) commonly have problems with backend processing due to a high percentage of insoluble solids in the thin stillage after the solid/liquid separation. Much of the thin stillage solids are fiber, proteins and polymeric sugars that contribute to the high percentage of insoluble solids and limit total solids in syrups, causing high viscosity issues in the evaporators and contribute to fouling.

WO 2002/38786 concerns ethanol ethanol processes wherein the viscosity of liquefied mash, thin stillage, condensate and/or syrup of evaporated thin stillage is reduced by addition of an effective amount of thinning enzymes selected from the group consisting of alpha-amylase, xylanase, xyloglucanase, cellulase, pectinase, or a mixture thereof.

It is desirable to provide fermentation product production processes that improves the use of recycled backset to the frontend of the process.

### BRIEF DESCRIPTION OF THE DRAWING

FIG. 1 schematically shows a dry grind ethanol production process.

FIG. 2 shows the effect of enzymatic hydrolysis on ethanol yield according to the invention.

### DESCRIPTION OF THE INVENTION

The invention relates to processes of producing fermentation products, especially ethanol, from starch-containing material where backset is recycled to the front-end of the process, in particular to the slurry tank.

Much of the thin stillage solids are fiber, proteins and polymeric sugars that contribute to the high percentage of insoluble solids and limit total solids in syrups, causing high viscosity issues in the evaporators and contribute to fouling. Reducing the thin stillage viscosity through hydrolysis of these insoluble solids would:
allow for the production of syrup with higher total solids content;
reduce evaporator fouling, and
increase the fermentation product yield.

The inventor has surprisingly found that when using selected enzymes for hydrolysing the thin stillage (i.e., hydrolysing the insoluble solids in the thin stillage) the backset can more efficiently be transported to the frontend of the process (e.g., slurry tank) resulting in reduced dependency on fresh water needed. Further, the fermentation product yield, i.e., ethanol yield, was also increased as shown in Example 1.

In the first aspect, the invention relates to processes of producing a fermentation product, in particular ethanol, from starch containing material comprising:

(a) forming a slurry comprising the starch-containing material and water;
(b) converting the starch-containing material into dextrins with an alpha-amylase;
(c) saccharifying the dextrins using a carbohydrate source generating enzyme to form sugars;
(d) fermenting sugars using a fermenting organism;
(e) recovering the fermentation product to form whole stillage;
(f) separating the whole stillage into a liquid fraction thin stillage and solid fraction wet cake;
(g) hydrolyzing the thin stillage;
(h) recycle a portion of the hydrolyzed thin stillage to steps (a);
wherein the thin stillage in step (g) is hydrolyzed using a glucoamylase and/or polygalactorunase.

The portion of the hydrolyzed thin stillage that is not recycled (i.e., as backset) in step (h) may be evaporated to syrup and condensate. In an embodiment the condensate is recycled to step (a).

In an embodiment the thin stillage is hydrolysed in step (g) at a temperature in the range from 20-80° C., such as in the range 30-70° C., in particular in the range 40-60° C., especially around 50° C. In an embodiment the dry solids (DS) content in the thin stillage is in the range from 10-50% (W/W), such as in the range from 20-45% (w/w) in particular 30-40% (w/w), especially around 35% (w/w). In an embodiment the thin stillage is hydrolysed in step (g) for 0.1-10 hours, such as 1-5 hours in particular around 2 hours.

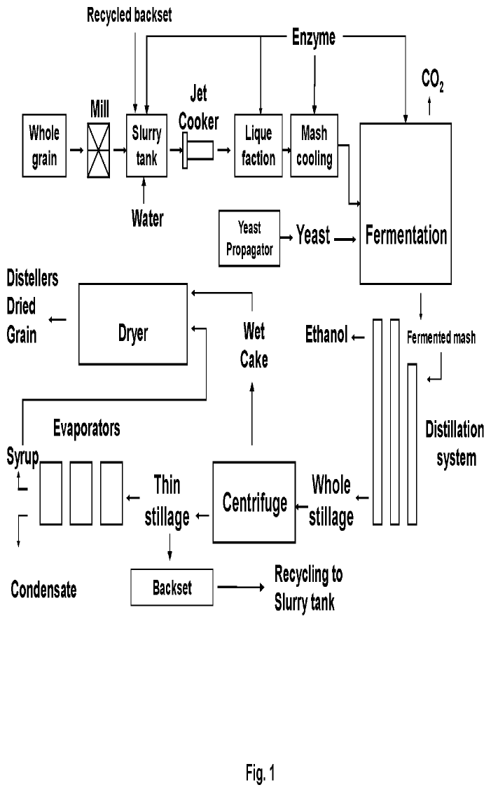

The process flow of a process of the invention may be similar or identical to that shown in FIG. 1 herein.

Between 5-90 vol-%, such as between 10-80%, such as between 15-70%, such as between 20-60% of the hydrolyzed thin stillage may be recycled (as backset) to step (a). The recycled hydrolyzed thin stillage (i.e., backset) may constitute from about 1-70 vol.-%, preferably 15-60% vol.-%, especially from about 30 to 50 vol.-% of the slurry formed in step (a).

Steps (a)-(d)

Prior to liquefying the starch-containing material into dextrins in step (b) with an alpha-amylase the particle size of the starch-containing material is reduced, preferably by milling, in particular dry milling (e.g. hammer milling) and a slurry comprising the starch-containing material and water is formed.

The aqueous slurry may contain from 10-55 wt.-% dry solids, preferably 25-45 wt. % dry solids, more preferably 30-40 wt.-% dry solids of starch-containing material.

The slurry in step (a) may be heated to above the initial gelatinization temperature and alpha-amylase, preferably bacterial alpha-amylase, in particular Bacillus stearothermophilus alpha-amylase, may be added. The temperature in step (a) may in an embodiment be between 40-60° C.

In an embodiment the slurry is jet-cooked before step (b), but after step (a), to gelatinize the slurry before being subjected to an alpha-amylase in step (b). Jet-cooking may be carried out at a temperature between 95-140° C. for about 1-15 minutes, preferably for about 3-10 minutes, especially around about 5 minutes.

The temperature in steps (b) is above the initial gelatinization temperature, such as between 70-100° C., such as between 80-95°, such as 85-93° C., such as about 88° C. or 91° C. Step (b) may typically be carried out for 0.1-12 hours, such as 1-5 hours.

In a preferred embodiment a protease is present in and/or added in steps (a) and/or step (b).

In an embodiment steps (a)-(b) are carried out as a three-step hot slurry process. The slurry is heated to between 70-100° C., preferably between 80-90° C., such as 85° C., or more preferably between 85° C. and 95° C., such as 88° or 91° C. Alpha-amylase may be added to initiate liquefaction (thinning). Then the slurry is jet-cooked at a temperature between 95-140° C., such as between 110-145° C., preferably between 120-140° C., preferably between 105-125° C., such as between 125-135° C., such as around 130° C., for 1-15 minutes, preferably for 3-10 minutes, especially around 5 minutes. The slurry is then cooled to 60-95° C., preferably 80-90° C., in particular around 85° C., and (more) alpha-amylase is added to finalize hydrolysis (secondary liquefaction), e.g., for 0.1-12 hours, such as 1-5 hours. The pH in steps (a) and/or (b) may be from 4-7, preferably 4.5-6.5, in particular between 5 and 6. Milled and liquefied starch-containing material is often referred to as “mash”.

The saccharification in step (c) may be carried out using conditions well-known in the art. For instance, saccharification may last up to from about 24 to about 72 hours. In an embodiment a pre-saccharification step (b′) is done for 40-90 minutes at a temperature between 30-65° C., typically at about 60° C., followed by complete saccharification during fermentation in a simultaneous saccharification and fermentation step (SSF). Saccharification is typically carried out at temperatures from 20-75° C., preferably from 40-70° C., such as around 60° C., and at a pH between 4 and 5, normally at about pH 4.5.

The most widely used process in fermentation product production, especially ethanol production, is simultaneous saccharification and fermentation (SSF), in which there is no holding stage for the saccharification. This means that the fermenting organism, such as yeast, and enzymes may be added together. Fermentation step (d) or simultaneous saccharification and fermentation (SSF) (i.e., steps (c) and (d)) are typically carried out at a temperature from 25° C. to 40° C., such as from 28° C. to 35° C., such as from 30° C. to 34° C., preferably around about 32° C. Fermentation step (d) or simultaneous saccharification and fermentation (SSF) (i.e., steps (c) and (d)) are typically ongoing for 6 to 120 hours, in particular 24 to 96 hours.

When producing ethanol the fermentation organism is typically yeast, such as a strain of Saccharomyces, in particular a strain of Saccharomyces cerevisiae.

Other fermentation products may be fermented at conditions and temperatures, well known to the skilled person in the art, suitable for the fermenting organism in question. According to the invention the temperature may be adjusted up or down during fermentation.

In an embodiment, a protease is adding during fermentation or SSF.

The fermentation product, such as especially ethanol, may be recovered after fermentation, e.g., by distillation.

### Starch-Containing Starting Materials

According to the invention any suitable starch-containing starting material may be used. The starting material is generally selected based on the desired fermentation product, here ethanol. Examples of starch-containing starting materials, suitable for use in processes of the present invention, include cereal, tubers or grains. Specifically the starch-containing material may be corn, wheat, barley, rye, milo, sago, cassava, tapioca, sorghum, oat, rice, peas, beans, or sweet potatoes, or mixtures thereof. Contemplated are also waxy and non-waxy types of corn and barley.

In a preferred embodiment the starch-containing starting material is corn.

In a preferred embodiment the starch-containing starting material is wheat.

In a preferred embodiment the starch-containing starting material is barley.

In a preferred embodiment the starch-containing starting material is rye.

In a preferred embodiment the starch-containing starting material is milo.

In a preferred embodiment the starch-containing starting material is sago.

In a preferred embodiment the starch-containing starting material is cassava.

In a preferred embodiment the starch-containing starting material is tapioca.

In a preferred embodiment the starch-containing starting material is sorghum.

In a preferred embodiment the starch-containing starting material is rice,

In a preferred embodiment the starch-containing starting material is peas.

In a preferred embodiment the starch-containing starting material is beans.

In a preferred embodiment the starch-containing starting material is sweet potatoes.

In a preferred embodiment the starch-containing starting material is oats.

### Fermentation

Fermentation is carried out in a fermentation medium. The fermentation medium includes the fermentation substrate, that is, the carbohydrate source that is metabolized by the fermenting organism. According to the invention the fermentation medium may comprise nutrients and growth stimulator(s) for the fermenting organism. Nutrient and growth stimulators are widely used in the art of fermentation and include nitrogen sources, such as ammonia; urea, vitamins and minerals, or combinations thereof.

### Fermenting Organisms

The term “fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 105 to 1012, preferably from 107 to 1010, especially about 5×107.

Examples of commercially available yeast includes, e.g., RED STAR™ and ETHANOL RED□ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H2 and CO2); antibiotics (e.g., penicillin and tetracycline); enzymes; vitamins (e.g., riboflavin, B12, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery of Fermentation Products

Subsequent to fermentation or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art.

### Enzymes Used for Hydrolysing Thin Stillage in Step (g)

According to the invention thin stillage is hydrolysed in step (g).

### Glucoamylase

In an embodiment the thin stillage is hydrolysed with a glucoamylase in step (g). The glucoamylase may be any glucoamylase, including for example, any of the glucoamylases added in steps (a), (b), (c), and (d), which are described below. In an embodiment the glucoamylase (E.C. 3.2.1.3) is a GH15 enzyme, in particular derived from the genus Trametes, such as Trametes cingulata, especially the one shown in SEQ ID NO: 1 herein.

In an embodiment the glucoamylase has at least 60%, at least 70%, at least 80%, at least 85%, at least 90%, at least 95%, at least 96%, at least 97%, at least 98%, at least 99%, such as 100% sequence identity to SEQ ID NO: 1 herein.

### Polygalacturonase

In an embodiment the thin stillage is hydrolysed in step (g) with a polygalacturonase (EC 3.2.1.15). Polygalacturonases are also known as endopolygalacturonase, endogalacturonase, endoD-galacturonase and are by the systematic name (1→4)-α-D-galacturonan glycanohydrolase (endo-cleaving). The enzyme catalyses the random hydrolysis of (1→4)-αD-galactosiduronic linkages in pectate and other galacturonans. Different forms of the enzyme have different tolerances to methyl esterification of the substrate.

The polygalacturonase may be any polygalacturonase. In an embodiment the polygalactunonase is derived from a strain of Aspergillus, for example a strain of Aspergillus aculeatus, Aspergillus fumigatus, Aspergillus kawachii, or Aspergillus niger, or Aspergillus tubigensis.

In an embodiment the polygalacturonase is the Aspergillus niger polygalacturonase shown in SEQ ID NO: 5 of WO2018/127486 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalacturonase is the Aspergillus aculeatus polygalacturonase shown in SEQ ID NO: 1017 of WO2018/204483 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalacturonase is the Aspergillus aculeatus polygalacturonase shown in SEQ ID NO: 17 of WO2020/002574 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalacturonase is the Aspergillus aculeatus polygalacturonase shown in SEQ ID NO: 7577 of WO2010/046471 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalacturonase is the Aspergillus tubigensis polygalacturonase described in WO2020/002574 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalacturonase is the Aspergillus tubigensis polygalacturonase described in WO1994/14966 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalacturonase is the Aspergillus aculeatus polygalacturonase shown in SEQ ID NO: 1018 of WO2018204483 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

In an embodiment the polygalactunonase is derived from a strain of Thermoascus, for example a strain of Thermoascus crustaceus.

In an embodiment the polygalacturonase is the Thermoascus crustaceus polygalacturonase shown in SEQ ID NO: 404 of WO2014/059541 (incorporated herein by reference in its entirety) or one having an amino acid sequence that has at least 60%, at least 70%, at least 80%, at least 85%, at least 90%, at least 95%, at least 96%, at least 97%, at least 98%, at least 99%, such as 100% sequence identity thereto.

### Alpha-Amylase

In an embodiment the thin stillage is further hydrolysed in step (g) with an alpha-amylase. The alpha-amylase may be any alpha-amylase. In an embodiment the alpha-amylase is a fungal acid alpha-amylase. In a preferred embodiment the alpha-amylase is derived from Rhizomucor, such as a strain of Rhizomucor pusillus, such as a Rhizomucor pusillus alpha-amylase with a starch-binding domain (SBD), such as a Rhizomucor pusillus alpha-amylase with linker and SBD, in particular Aspergillus niger glucoamylase and linker. In a preferred embodiment the alpha-amylase is the one shown in SEQ ID NO: 2 herein.

In an embodiment the alpha-amylase has at least 60%, at least 70%, at least 80%, at least 85%, at least 90%, at least 95%, at least 96%, at least 97%, at least 98%, at least 99%, such as 100% sequence identity to SEQ ID NO: 2 herein.

In an embodiment the alpha-amylase is a variant of the alpha-amylase shown in SEQ ID NO: 2 herein having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 2 for numbering).

In a preferred embodiment the alpha-amylase is derived from Rhizomucor pusillus with an Aspergillus niger glucoamylase linker and starch-binding domain (SBD), preferably the one disclosed as SEQ ID NO: 2 herein, preferably having one or more of the following substitutions: G128D, D143N, preferably G128D+D143N (using SEQ ID NO: 2 for numbering).

In an embodiment the alpha-amylase variant has at least 70%, such as at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 herein.

### Pullulanase

In an embodiment the thin stillage is further hydrolysed in step (g) with a pullulanase (E.C. 3.2.1.41). The pullulanase may be any pullulanase. In an embodiment the pullulanase is derived from a strain of Bacillus, such as Bacillus deramificans, in particular the one shown in SEQ ID NO: 3 herein.

In an embodiment the pullulanase has at least 60%, at least 70%, at least 80%, at least 85%, at least 90%, at least 95%, at least 96%, at least 97%, at least 98%, at least 99%, such as 100% sequence identity to SEQ ID NO: 3 herein.

### Laminarinase

In an embodiment the thin stillage is hydrolysed in step (g) with a laminarinase (E.C. 3.2.1.6). The laminarinase may be any laminarinase. In an embodiment the laminarinase is derived from a strain of Aspergillus, such as a strain of Aspergillus aculeatus.

### Combination of Enzymes Used for Hydrolyzing Thin Stillage in Step (q)

According to the invention thin stillage is hydrolysed with a combination of enzymes in step (g).

In a preferred embodiment the thin stillage is hydrolysed in step (g) with a combination of glucoamylase and alpha-amylase, such as the one mentioned above, in particular the glucoamylase shown in SEQ ID NO: 1 and the alpha-amylase shown in SEQ ID NO: 2 having the following substitutions: G128D+D143N.

In an embodiment the thin stillage is hydrolysed in step (g) with a combination of glucoamylase and pullulanase.

In an embodiment the thin stillage is hydrolysed in step (g) with a combination of polygalacturonase and laminarinase.

Alpha-Amylase Present and/or Added in Step (a) and/or Step (b)

According to the invention an alpha-amylase is present and/or added in step (a) and/or step (b). The alpha-amylase present and/or added in step (a) and/or step (b) may be any alpha-amylase. Preferred are bacterial alpha-amylases, which typically are stable at high temperatures.

### Bacterial Alpha-Amylase

The term “bacterial alpha-amylases” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase of SEQ ID NO: 3 in WO 99/19467 or SEQ ID NO: 4 herein, the Bacillus amyloliquefaciens alpha-amylase of SEQ ID NO: 5 in WO 99/19467, and the Bacillus licheniformis alpha-amylase of SEQ ID NO: 4 in WO 99/19467 (all sequences are hereby incorporated by reference). In an embodiment the alpha-amylase has at least 60%, e.g., at least 70%, at least 80%, at least 90%, at least 95%, at least 96%, at least 97%, at least 98% at least 99% or 100% sequence identity to any of the sequences shown in SEQ ID NOS: 3, 4 or 5, respectively, in WO 99/19467.

In an embodiment the alpha-amylase has at least 60%, e.g., at least 70%, at least 80%, at least 90%, at least 91%, at least 92%, at least 93%, at least 94%, at least 95%, at least 96%, at least 97%, at least 98% or at least 99%, or 100% sequence identity to the mature part of SEQ ID NO: 4 herein.

In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated so it is between 485 and 495 amino acids long, such as around 491 amino acids long, e.g., so that it lacks a functional starch binding domain (compared to SEQ ID NO: 3 in WO 99/19467) or SEQ ID NO: 4 herein.

The Bacillus alpha-amylase may also be a variant and/or hybrid. Examples of such a variant can be found in any of WO 96/23873, WO 96/23874, WO 97/41213, WO 99/19467, WO 00/60059, and WO 02/10355 (all documents are hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, I181 and/or G182, preferably a double deletion disclosed in WO 96/23873—see, e.g., page 20, lines 1-10 (hereby incorporated by reference), preferably corresponding to deletion of positions I181 and G182 compared to the amino acid sequence of Bacillus stearothermophilus alpha-amylase set forth in SEQ ID NO: 3 disclosed in WO 99/19467 or SEQ ID NO: 4 herein or the deletion of amino acids R179 and G180 using SEQ ID NO: 3 in WO 99/19467 or SEQ ID NO: 4 herein for numbering (which reference is hereby incorporated by reference). Even more preferred are Bacillus alpha-amylases, especially Bacillus stearothermophilus alpha-amylases, which have a double deletion corresponding to a deletion of positions 181 and 182, and optionally further comprises a N193F substitution (also denoted I181*+G182*+N193F) compared to the wild-type BSG alpha-amylase amino acid sequence set forth in SEQ ID NO: 3 disclosed in WO 99/19467 or SEQ ID NO: 4 herein. The bacterial alpha-amylase may also have a substitution in a position corresponding to S239 in the Bacillus licheniformis alpha-amylase shown in SEQ ID NO: 4 in WO 99/19467, or a S242 and/or E188P variant of the Bacillus stearothermophilus alpha-amylase of SEQ ID NO: 3 in WO 99/19467 or SEQ ID NO: 4 herein.

In an embodiment the variant is a S242A, E or Q variant, preferably a S242Q variant, of the Bacillus stearothermophilus alpha-amylase (using SEQ ID NO: 4 herein for numbering).

In an embodiment the variant is a position E188 variant, preferably E188P variant of the Bacillus stearothermophilus alpha-amylase (using SEQ ID NO: 4 herein for numbering).

In an embodiment of the invention the bacterial alpha-amylase, preferably derived from the genus Bacillus, especially a strain of Bacillus stearothermophilus, in particular the Bacillus stearothermophilus as disclosed in WO 99/019467 as SEQ ID NO: 3 or SEQ ID NO: 4 herein with one or two amino acids deleted at positions R179, G180, I181 and/or G182, in particular with R179 and G180 deleted, or with I181 and G182 deleted, further with mutations from below list of mutations.

In preferred embodiments the Bacillus stearothermophilus alpha-amylase has a I181+G182 double deletion, and optional a N193F substitution, and further comprises mutations selected from below list:

V59A + Q89R + G112D + E129V + K177L + R179E + K220P + N224L + Q254S;
V59A + Q89R + E129V + K177L + R179E + H208Y + K220P + N224L + Q254S;
V59A + Q89R + E129V + K177L + R179E + K220P + N224L + Q254S + D269E + D281N;
V59A + Q89R + E129V + K177L + R179E + K220P + N224L + Q254S + I270L;
V59A + Q89R + E129V + K177L + R179E + K220P + N224L + Q254S + H274K;
V59A + Q89R + E129V + K177L + R179E + K220P + N224L + Q254S + Y276F;
V59A + E129V + R157Y + K177L + R179E + K220P + N224L + S242Q + Q254S;
V59A + E129V + K177L + R179E + H208Y + K220P + N224L + S242Q + Q254S;
59A + E129V + K177L + R179E + K220P + N224L + S242Q + Q254S;
V59A + E129V + K177L + R179E + K220P + N224L + S242Q + Q254S + H274K;
V59A + E129V + K177L + R179E + K220P + N224L + S242Q + Q254S + Y276F;
V59A + E129V + K177L + R179E + K220P + N224L + S242Q + Q254S + D281N;
V59A + E129V + K177L + R179E + K220P + N224L + S242Q + Q254S + M284T;
V59A + E129V + K177L + R179E + K220P + N224L + S242Q + Q254S + G416V;
V59A + E129V + K177L + R179E + K220P + N224L + Q254S;
V59A + E129V + K177L + R179E + K220P + N224L + Q254S + M284T;
A91L + M96I + E129V + K177L + R179E + K220P + N224L + S242Q + Q254S;
E129V + K177L + R179E;
E129V + K177L + R179E + K220P + N224L + S242Q + Q254S;
E129V + K177L + R179E + K220P + N224L + S242Q + Q254S + Y276F + L427M;
E129V + K177L + R179E + K220P + N224L + S242Q + Q254S + M284T;
E129V + K177L + R179E + K220P + N224L + S242Q + Q254S + N376* + I377*;
E129V + K177L + R179E + K220P + N224L + Q254S;
E129V + K177L + R179E + K220P + N224L + Q254S + M284T;
E129V + K177L + R179E + S242Q;
E129V + K177L + R179V + K220P + N224L + S242Q + Q254S;
K220P + N224L + S242Q + Q254S;
M284V;
V59A + Q89R + E129V + K177L + R179E + Q254S + M284V.
V59A + E129V + K177L + R179E + Q254S + M284V;

In a preferred embodiment the alpha-amylase is selected from the group of Bacillus stearothermophilus alpha-amylase variants:
I181*+G182*+N193F+E129V+K177L+R179E;
181*+G182*+N193F+V59A+Q89R+E129V+K177L+R179E+H208Y+K220P+N224L+Q254S;
I181*+G182*+N193F+V59A+Q89R+E129V+K177L+R179E+Q254S+M284V;
I181*+G182*+N193F+V59A+E129V+K177L+R179S+Q254S+M284V
I181*+G182*+N193F+V59A+E129V+K177L+R179E+Q254S+M284V; and
I181*+G182*+N193F+E129V+K177L+R179E+K220P+N224L+S242Q+Q254S (using SEQ ID NO: 4 herein for numbering).

It should be understood that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 4 herein, or variants thereof, are truncated in the C-terminal and are typically around 491 amino acids long, such as from 480-495 amino acids long, or so that it lacks a functional starch binding domain.

In a preferred embodiment the alpha-amylase variant may be an enzyme having at least 60%, e.g., at least 70%, at least 80%, at least 90%, at least 95%, at least 91%, at least 92%, at least 93%, at least 94%, at least 95%, at least 96%, at least 97%, at least 98% or at least 99%, but less than 100% sequence identity to the sequence shown in SEQ ID NO: 3 in WO 99/19467 or SEQ ID NO: 4 herein.

In an embodiment the bacterial alpha-amylase, e.g., Bacillus alpha-amylase, such as especially Bacillus stearothermophilus alpha-amylase, or variant thereof, is dosed to liquefaction in a concentration between 0.01-10 KNU-A/g DS, e.g., between 0.02 and 5 KNU-A/g DS, such as 0.03 and 3 KNU-A, preferably 0.04 and 2 KNU-A/g DS, such as especially 0.01 and 2 KNU-A/g DS. In an embodiment the bacterial alpha-amylase, e.g., Bacillus alpha-amylase, such as especially Bacillus stearothermophilus alpha-amylases, or variant thereof, is dosed to step (a) and/or (b) in a concentration of between 0.0001-1 mg EP (Enzyme Protein)/g DS, e.g., 0.0005-0.5 mg EP/g DS, such as 0.001-0.1 mg EP/g DS.

Protease Present and/or Added in Liquefaction

According to the invention a protease is optionally present and/or added in step (a) and/or step (b) together with an alpha-amylase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). See Assay in the “Materials & Methods” section

In one embodiment the protease is of fungal origin.

The protease may be a variant of, e.g., a wild-type protease. In a preferred embodiment the protease is a thermostable variant of a metallo protease. In an embodiment the thermostable alpha-amylase used in a process of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of Thermoascus aurantiacus CGMCC No. 0670 protease. Suitable protease variants are disclosed in WO 2011/072191, including the variant disclosed in Tables 1-6 in Example 1 (which are hereby incorporated by reference. In a preferred embodiment the protease is a thermostable variant of the mature part of the metallo protease shown as SEQ ID NO: 1 in WO 2010/008841 and shown as SEQ ID NO: 7 herein further with mutations selected from below list:

### D79L+S87P+A112P+D142L;

### D79L+S87P+D142L; or

### A27K+D79L+Y82F+S87G+D104P+A112P+A126V+D142L.

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 1 in WO 2010/008841 or SEQ ID NO: 7 herein.

In one embodiment the protease is of bacterial origin.

In a preferred embodiment the protease is a thermostable protease derived from a strain of the bacterium Pyrococcus, such as a strain of Pyrococcus furiosus.

In an embodiment the protease is one shown as SEQ ID NO: 1 in U.S. Pat. No. 6,358,726-B1 (Takara Shuzo Company), or SEQ ID NO: 8 herein.

In another embodiment the (thermostable) protease is one disclosed in SEQ ID NO: 8 herein or a protease having at least 70%, such as at least 80%, such as at least 85%, such as at least 90%, such as at least 95%, such as at least 96%, such as at least 97%, such as at least 98%, such as at least 99% or 100% sequence identity to SEQ ID NO: 1 in U.S. Pat. No. 6,358,726-B1 or SEQ ID NO: 8 herein.

Glucoamylase Present and/or Added in Step (a) and/or Step (b)

According to the invention a glucoamylase may optionally be present and/or added in step (a) and/or step (b). In a preferred embodiment the glucoamylase is added together with or separately from the alpha-amylase and/or the protease. In an embodiment the glucoamylase is a thermostable glucoamylase, e.g., one having a Relative Activity heat stability at 85° C. of at least 20%, at least 30%, preferably at least 35% determined as described in Example 4 (heat stability) in WO 2011/127802 (hereby incorporated by reference).

In a preferred embodiment the glucoamylase is one derived from a strain of Penicillium, e.g., the one show in SEQ ID NO: 9 herein.

Contemplated Penicillium oxalicum glucoamylase variants of SEQ ID NO: 9 herein include the ones disclosed in WO 2013/053801 which is hereby incorporated by reference. Specific examples include glucoamylase variants comprising at least one of the following combinations of substitutions:

### P11F+T65A+Q327F; or

### P2N+P4S+P11F+T65A+Q327F; or

### P11F+D26C+K33C+T65A+Q327F; or

### P2N+P4S+P11F+T65A+Q327W+E501V+Y504T; or

### P2N+P4S+P11F+T65A+Q327F+E501V+Y504T; or

### P11F+T65A+Q327W+E501V+Y504T.

The glucoamylase may be added in amounts from 0.1-100 micrograms EP/g, such as 0.5-50 micrograms EP/g, such as 1-25 micrograms EP/g, such as 2-12 micrograms EP/g DS.

Carbohydrate-Source Generating Enzyme Present and/or Added During Saccharification Step (c) and/or Fermentation Step (d)

According to the invention a carbohydrate-source generating enzyme is present and/or added during saccharification step (c) and/or fermentation step (d).

In a preferred embodiment the carbohydrate-source generating enzyme is a glucoamylase, of fungal origin, preferably from a stain of Aspergillus, preferably A. niger, A. awamori, or A. oryzae; or a strain of Trichoderma, preferably T. reesei; or a strain of Talaromyces, preferably T. emersonii, or a strain of Gloephyllum, preferably G. sepiarium or G. trabeum; or a strain of Pycnoporus, preferably Pycnoporus sanguineus.

### Glucoamylase

According to the invention the glucoamylase present and/or added during saccharification step (b) and/or fermentation step (d) may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). In a preferred embodiment the glucoamylase used during saccharification and/or fermentation is the Talaromyces emersonii glucoamylase disclosed in WO 99/28448 as SEQ ID NO: 34 (hereby incorporated by reference.

Contemplated fungal glucoamylases include Trametes cingulata, Pachykytospora papyracea; and Leucopaxillus giganteus all disclosed in WO 2006/069289; and Peniophora rufomarginata disclosed in WO2007/124285; or a mixture thereof. Also hybrid glucoamylase are contemplated according to the invention. Examples include the hybrid glucoamylases disclosed in WO 2005/045018. Specific examples include the hybrid glucoamylase disclosed in Table 1 and 4 of Example 1 (which hybrids are hereby incorporated by reference).

In an embodiment the glucoamylase is derived from a strain of the genus Pycnoporus, in particular a strain of Pycnoporus as described in as WO 2011/066576 (SEQ ID NOs 2, 4 or 6), or from a strain of the genus Gloephyllum, in particular a strain of Gloephyllum as described in WO 2011/068803 (SEQ ID NO: 2, 4, 6, 8, 10, 12, 14 or 16) or a strain of the genus Nigrofomes, in particular a strain of Nigrofomes sp. disclosed in WO 2012/064351 as SEQ ID NO: 2 (all references hereby incorporated by reference).

Contemplated are also glucoamylases which have at least 60%, such as at least 70%, at least 75%, at least 80%, at least 85%, at least 90%, at least 95%, at least 96%, at least 97%, at least 98%, at least 99% or even 100% sequence identity to any one of the mature parts of the enzyme sequences mentioned above.

In an embodiment the glucoamylase present and/or added to the saccharification step (c) and/or fermentation step (d) further comprising an alpha-amylase. In a preferred embodiment the alpha-amylase is a fungal alpha-amylase, especially an acid fungal alpha-amylase.

In an embodiment the glucoamylase is a blend comprising Talaromyces emersonii glucoamylase disclosed in WO 99/28448 as SEQ ID NO: 34 and Trametes cingulata glucoamylase disclosed as SEQ ID NO: 2 in WO 2006/069289 and SEQ ID NO: 1 herein.

In an embodiment the glucoamylase is a blend comprising Talaromyces emersonii glucoamylase disclosed in WO 99/28448 as SEQ ID NO: 34, Trametes cingulata glucoamylase disclosed as SEQ ID NO: 2 in WO 06/69289 and SEQ ID NO: 1 herein, and Rhizomucor pusillus alpha-amylase with Aspergillus niger glucoamylase linker and SBD disclosed as V039 in Table 5 in WO 2006/069290 or SEQ ID NO: 2 herein.

In an embodiment the glucoamylase is a blend comprising Talaromyces emersonii glucoamylase disclosed in WO 99/28448 as SEQ ID NO: 34, Trametes cingulata glucoamylase disclosed in WO 2006/69289 and as SEQ ID NO: 1 herein, and Rhizomucor pusillus alpha-amylase with Aspergillus niger glucoamylase linker and SBD disclosed as V039 in Table 5 in WO 2006/069290 or SEQ ID NO: 2 herein.

In an embodiment the glucoamylase is a blend comprising Gloeophyllum sepiarium glucoamylase shown as SEQ ID NO: 2 in WO 2011/068803 and Rhizomucor pusillus with an Aspergillus niger glucoamylase linker and starch-binding domain (SBD), disclosed SEQ ID NO: 3 in WO 2013/006756 and SEQ ID NO: 2 herein with the following substitutions: G128D+D143N.

Contemplated are also embodiment where the alpha-amylase is derived from a strain of the genus Rhizomucor, preferably a strain the Rhizomucor pusillus, such as the one shown in SEQ ID NO: 3 in WO2013/006756, or the Rhizomucor pusillus alpha-amylase with an Aspergillus niger glucoamylase linker and starch-binding domain (SBD) has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 3 in WO 2013/006756 for numbering or SEQ ID NO: 2 herein).

In a preferred embodiment the glucoamylase blend comprises Gloeophyllum sepiarium glucoamylase (e.g., SEQ ID NO: 2 in WO 2011/068803 or SEQ ID NO: 15 herein) and Rhizomucor pusillus alpha-amylase.

In a preferred embodiment the glucoamylase blend comprises Gloeophyllum sepiarium glucoamylase shown as SEQ ID NO: 2 in WO 2011/068803 or SEQ ID NO: 15 herein and Rhizomucor pusillus with an Aspergillus niger glucoamylase linker and starch-binding domain (SBD), disclosed SEQ ID NO: 3 in WO 2013/006756 and SEQ ID NO: 16 herein with the following substitutions: G128D+D143N.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available products comprising glucoamylase include AMG 200L; AMG 300 L; SAN™ ′ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE™ and AMG™ E (from Novozymes A/S).

Cellulolytic Composition Present and/or Added During Saccharification Step (c) and/or Fermentation Step (d)

According to the invention a cellulolytic composition may be present and/or added in saccharification step (c), fermentation step (d) or simultaneous saccharification and fermentation (SSF).

The cellulolytic composition comprises a beta-glucosidase, a cellobiohydrolase and an endoglucanase.

Examples of suitable cellulolytic composition can be found in WO 2008/151079, WO 2011/057140 and WO 2013/028928 which are incorporated by reference.

In embodiments the cellulolytic composition is derived from a strain of Trichoderma, Humicola, or Chrysosporium.

In preferred embodiments the cellulolytic composition is derived from a strain of Trichoderma reesei, Humicola insolens and/or Chrysosporium lucknowense.

In a preferred embodiment the cellulolytic composition is derived from a strain of Trichoderma reesei.

In an embodiment the cellulolytic composition is dosed from 0.0001-3 mg EP/g DS, preferably, 0.0005-2 mg EP/g DS, preferably 0.001-1 mg/g DS, more preferably 0.005-0.5 mg EP/g DS, and even more preferably 0.01-0.1 mg EP/g DS.

The invention is further summarized in the following paragraphs:

1. A process of producing a fermentation product from starch containing material comprising:
(a) forming a slurry comprising the starch-containing material and water;
(b) converting the starch-containing material into dextrins with an alpha-amylase;
(c) saccharifying the dextrins using a carbohydrate source generating enzyme to form sugars;
(d) fermenting sugars using a fermenting organism;
(e) recovering the fermentation product to form whole stillage;
(f) separating the whole stillage into a liquid fraction thin stillage and solid fraction wet cake;
(g) hydrolyzing the thin stillage;
(h) recycle a portion of the hydrolyzed thin stillage to steps (a);
wherein the thin stillage in step (g) is hydrolyzed using a glucoamylase and/or polygalactorunase.
2. The process of paragraph 1, wherein the portion of the hydrolyzed thin stillage that is not recycled (i.e., as backset) is evaporated to syrup and condensate.
3. The process of paragraph 2, wherein the condensate is recycled to step (a).
4. The process of any of paragraphs 1-3, wherein between 5-90 vol-%, such as between 10-80%, such as between 15-70%, such as between 20-60% of the hydrolyzed thin stillage is recycled as backset to step (a).
5. The process of any of paragraphs 1-4, wherein the recycled hydrolyzed thin stillage (i.e., backset) constitutes from about 1-70 vol.-%, preferably 15-60% vol.-%, especially from about 30 to 50 vol.-% of the slurry formed in step (a).
6. The process of any of paragraphs 1-5, wherein steps (c) and (d) are carried out simultaneously or sequentially.
7. The process of any of paragraphs 1-6, wherein alpha-amylase is added in step (a).
8. The process of any of paragraphs 1-7, wherein the thin stillage is hydrolyzed in step (g) with a a glucoamylase (E.C. 3.2.1.3), preferably a GH15 enzyme, in particular derived from the genus Trametes, such as Trametes cingulata, especially the one shown in SEQ ID NO: 1 herein.
9. The process of paragraph 8, wherein the glucoamylase has at least 60%, at least 70%, at least 80%, at least 85%, at least 90%, at least 95%, at least 96%, at least 97%, at least 98%, at least 99%, such as 100% sequence identity to SEQ ID NO: 1 herein.
10. The process of any of paragraphs 1-9, further wherein the thin stillage is hydrolysed in step (g) with an alpha-amylase, in particular fungal acid alpha-amylase activity, such as a Rhizomucor alpha-amylase, such as a strain of Rhizomucor pusillus, such as a Rhizomucor pusillus alpha-amylase with a starch-binding domain (SBD), such as a Rhizomucor pusillus alpha-amylase with linker and SBD, in particular Aspergillus niger glucoamylase linker and SBD, specifically the alpha-amylase shown as SEQ ID NO: 2 herein.
11. The process of paragraph 10, wherein the fungal acid alpha-amylase has at least 60%, at least 70%, at least 80%, at least 85%, at least 90%, at least 95%, at least 96%, at least 97%, at least 98%, at least 99%, such as 100% sequence identity to SEQ ID NO: 2 herein.
12. The process of any of paragraphs 1-11, wherein the polygalacturonase (EC 3.2.1.15) used for hydrolysing the thin stillage in step (g) is preferably derived from a strain of Aspergillus, in particular a strain of Aspergillus aculeatus.
13. The process of any of paragraphs 1-12, further wherein the thin stillage is hydrolysed in step (g) with a pullulanase (E.C. 3.2.1.41), in particular derived from a strain of Bacillus, such as Bacillus deramificans, in particular the one shown in SEQ ID NO: 3 herein.
14. The process of paragraph 13, wherein the pullulanase has at least 60%, at least 70%, at least 80%, at least 85%, at least 90%, at least 95%, at least 96%, at least 97%, at least 98%, at least 99%, such as 100% sequence identity to SEQ ID NO: 3 herein.
15. The process of any of paragraphs 1-14, further wherein the thin stillage is hydrolysed in step (g) with a laminarinase (E.C. 3.2.1.6), in particular derived from a strain of Aspergillus, such as a strain of Aspergillus, for example a strain of Aspergillus aculeatus, Aspergillus fumigatus, Aspergillus kawachii, or Aspergillus niger, or Aspergillus tubigensis, or derived from a strain of Thermoascus, for example, Thermoascus crustaceus.
16. The process of any of paragraphs 1-15, wherein the thin stillage is hydrolysed in step (g) with a combination of glucoamylase and alpha-amylase.
17. The process of any of paragraphs 1-16, wherein the thin stillage is hydrolysed in step (g) with a combination of glucoamylase and pullulanase.
18. The process of any of paragraphs 1-17, wherein the thin stillage is hydrolysed in step (g) with a combination of polygalacturonase and laminarinase.
19. The process of any of paragraphs 1-18, wherein the thin stillage is hydrolysed in step (g) at a temperature in the range from 20-80° C., such as in the range 30-70° C., in particular in the range 40-60° C., especially around 50° C.
20. The process of any of paragraphs 1-19, wherein the dry solids (DS) content in the thin stillage is in the range from 10-50% (W/W), such as in the range from 20-45% (w/w) in particular 30-40% (w/w), especially around 35% (w/w).
21. The process of any of paragraphs 1-20, wherein the thin stillage is hydrolysed in step (g) for 0.1-10 hours, such as 1-5 hours in particular around 2 hours.
22. The process of any of paragraphs 1-21, wherein the process flow is similar or identical to that shown in FIG. 1 herein.
23. The process of any of paragraphs 1-22, wherein a protease is present in and/or added in steps (a) and/or (b).
24. The process of any of paragraphs 1-23, wherein the temperature in step (b) is above the initial gelatinization temperature, such as at a temperature between 70-100° C., such as between 80-90° C., such as around 85° C.
25. The process of any of paragraphs 1-24, wherein a jet-cooking step is carried out before step (b) and after step (a).
26. The process of paragraph 25, wherein jet-cooking is carried out at a temperature between 95-140° C. for about 1-15 minutes, preferably for about 3-10 minutes, especially around about 5 minutes.
27. The process of any of paragraph 1-26, wherein the pH in steps (a) and/or (b) is from 4-7, preferably 4.5-6.5, in particular between 5 and 6
28. The process of any of paragraphs 1-27, wherein the temperature in step (a) is 40-60° C.
29. The process of any of paragraphs 1-29, further comprising, before step (a), the steps of: reducing the particle size of the starch-containing material, preferably by dry milling (e.g., by hammer milling).
30. The process of any of paragraphs 1-29, further comprising a pre-saccharification step (b′), before saccharification step (c), carried out for 40-90 minutes at a temperature between 30-65° C.
31. The process of any of paragraphs 1-30, wherein saccharification in step (c) is carried out at a temperature from 20-75° C., preferably from 40-70° C., such as around 60° C., and at a pH between 4 and 5.
32. The process of any of paragraphs 1-31, wherein fermentation step (d) or simultaneous saccharification and fermentation (SSF) (i.e., steps (c) and (d)) are carried out at a temperature from 25° C. to 40° C., such as from 28° C. to 35° C., such as from 30° C. to 34° C., preferably around about 32° C.
33. The process of any of paragraphs 1-32, wherein fermentation step (d) or simultaneous saccharification and fermentation (SSF) (i.e., steps (c) and (d)) are ongoing for 6 to 120 hours, in particular 24 to 96 hours.
34. The process of any of paragraphs 1-33, wherein step (b) (i.e., liquefaction) is carried out for 0.1-12 hours, such as 1-5 hours.
35. The process of any of paragraphs 1-34, wherein step (b) (i.e., liquefaction) is carried our using a bacterial alpha-amylase, such as a bacterial alpha-amylase, in particular a Bacillus stearothermophilus alpha-amylase, such as the one shown in SEQ ID NO: 4 herein or a variant thereof.
36. The process of any of paragraphs 1-35, wherein separation in step (f) is carried out by centrifugation, preferably a decanter centrifuge, filtration, preferably using a filter press, a screw press, a plate-and-frame press, a gravity thickener or decker.
37. The process of any of paragraphs 1-36, wherein the starch-containing material is cereal.
38. The process of any of paragraphs 1-37, wherein the starch-containing material is selected from the group consisting of corn, wheat, barley, cassava, sorghum, rye, potato, beans, milo, peas, rice, sago, sweet potatoes, tapioca, oats or any combination thereof.
39. The process of any of paragraphs 1-38, wherein the fermentation product is selected from the group consisting of alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H2 and CO2), and more complex compounds, including, for example, antibiotics (e.g., penicillin and tetracycline); enzymes; vitamins (e.g., riboflavin, B12, beta-carotene); and hormones.
40. The process of any of paragraphs 1-39, wherein the fermentation product is ethanol.
The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

### Material & Methods

Glucoamylase Blend 10 (GAB10) is a blend of Trametes cingulata glucoamylase (SEQ ID NO: 1 herein) and Rhizomucor pusillus alpha-amylase (SEQ ID NO: 2 herein) (ratio about 10:1)
Glucoamylase TC (GATC): Trametes cingulata glucoamylase (SEQ ID NO: 1 herein)
Glucoamylase DX (GADX): Aspergillus niger glucoamylase (SEQ ID NO: 5 herein) and Bacillus deramificans pullulanase (SEQ ID NO: 3 herein) (AGU: NPUN ratio 1:2)
Laminarinase AC (LAC): Aspergillus aculeatus laminarinase (E.C. 3.2.1.6) with polygalacturonase and hemicellulose side activity.
Polygalacturonase UF (PGUF): Aspergillus aculeatus polygalacturonase.

### Yeast:

ETHANOL RED™: Saccharomyces cerevisiae yeast available from Fermentis/Lesaffre, USA.

### Methods

Identity: The relatedness between two amino acid sequences or between two nucleotide sequences is described by the parameter “identity”.
For purposes of the present invention the degree of identity between two amino acid sequences, as well as the degree of identity between two nucleotide sequences, may be determined by the program “align” which is a Needleman-Wunsch alignment (i.e. a global alignment). The program is used for alignment of polypeptide, as well as nucleotide sequences. The default scoring matrix BLOSUM50 is used for polypeptide alignments, and the default identity matrix is used for nucleotide alignments. The penalty for the first residue of a gap is −12 for polypeptides and −16 for nucleotides. The penalties for further residues of a gap are −2 for polypeptides, and −4 for nucleotides.
“Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,”
Methods in Enzymology 183:63-98). FASTA protein alignments use the Smith-Waterman algorithm with no limitation on gap size (see “Smith-Waterman algorithm”, T. F. Smith and M. S. Waterman (1981) J. Mol. Biol. 147:195-197).

### Protease Assays

### AZCL-Casein Assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

### Glucoamylase Activity (AGU)

Glucoamylase activity may be measured in Glucoamylase Units (AGU).
The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.
An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | | |
|---|---|---|
| Substrate: | maltose 23.2 mM | |
| Buffer: | acetate 0.1M | |
| pH: | 4.30 ± 0.05 | |
| Incubation | 37° C. ± 1 | |
| temperature: | | |
| Reaction time: | 5 | minutes |
| Enzyme working | 0.5-4.0 | AGU/mL |
| range: | | |

| Color reaction: | | |
|---|---|---|
| GlucDH: | 430 | U/L |
| Mutarotase: | 9 | U/L |
| NAD: | 0.21 | mM |
| Buffer: | phosphate 0.12M; | |
| | 0.15M NaCl | |
| pH: | 7.60 ± 0.05 | |
| Incubation | 37° C. ± 1 | |
| temperature: | | |
| Reaction time: | 5 | minutes |
| Wavelength: | 340 | nm |

A folder (EB-SM-0131.02/01) describing this analytical method in more detail is available on request from Novozymes A/S, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucanglucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

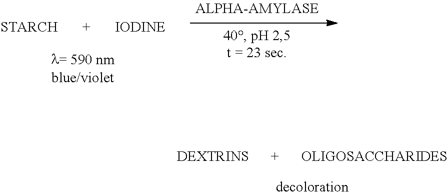

Standard conditions/reaction conditions:

Substrate: Soluble starch, approx. 0.17 g/L

Buffer: Citrate, approx. 0.03 M

Iodine (12): 0.03 g/L

CaCl2: 1.85 mM

pH: 2.50±0.05

Incubation temperature: 40° C.

Reaction time: 23 seconds

Wavelength: 590 nm

Enzyme concentration: 0.025 AFAU/mL

Enzyme working range: 0.01-0.04 AFAU/mL

A folder EB-SM-0259.02/01 describing this analytical method in more detail is available upon request to Novozymes A/S, Denmark, which folder is hereby included by reference.

Alpha-amylase activity (KNU)
The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.
One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.
A folder EB-SM-0009.02/01 describing this analytical method in more detail is available upon request to Novozymes A/S, Denmark, which folder is hereby included by reference.

### Alpha-Amylase Activity (KNU-A)

Alpha amylase activity is measured in KNU(A) Kilo Novozymes Units (A), relative to an enzyme standard of a declared strength.

Alpha amylase in samples and α-glucosidase in the reagent kit hydrolyze the substrate (4,6-ethylidene(G₇)-p-nitrophenyl(G₁)-α,D-maltoheptaoside (ethylidene-G₇PNP) to glucose and the yellow-colored p-nitrophenol.

The rate of formation of p-nitrophenol can be observed by Konelab 30. This is an expression of the reaction rate and thereby the enzyme activity.

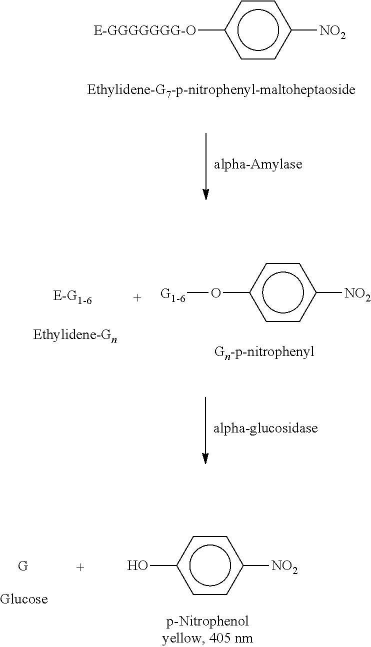

The enzyme is an alpha-amylase with the enzyme classification number EC 3.2.1.1.

| Parameter | Reaction conditions | |
|---|---|---|
| Temperature | 37° | C. |
| pH | 7.00 (at 37° C.) | |
| Substrate conc. | Ethylidene-G₇PNP, R2: 1.86 mM | |
| Enzyme conc. | 1.35-4.07 | KNU(A)/L |
| (conc. of high/low standard | | |
| in reaction mixture) | | |
| Reaction time | 2 | min |
| Interval kinetic measuring time | 7/18 | sec. |
| Wave length | 405 | nm |

| | |
|---|---|
| Conc. of reagents/chemicals | α-glucosidase, |
| critical for the analysis | R1: ≥3.39 kU/L |

A folder EB-SM-5091.02-D on determining KNU-A activity is available upon request to Novozymes A/S, Denmark, which folder is hereby included by reference.

### Determination of FAU(F)

FAU(F) Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 mL diluted sample or standard is incubated at 40° C. for 2 minutes. 0.5 mL 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

The present invention is described in further detail in the following examples which are offered to illustrate the present invention, but not in any way intended to limit the scope of the invention as claimed. All references cited herein are specifically incorporated by reference for that which is described therein.

### Example 1

This experiment investigates the effect of using enzymatically hydrolyzed thin stillage on ethanol yield when recycled as backset to the front end of an ethanol process

### Experimental Procedures:

Industrially produced condensate syrup (i.e., evaporated thin stillage) from a dry-grind ethanol plant was supplemented with 3 ppm penicillin and 500 ppm urea and adjusted to pH 5 with 40% v/v H2SO4. A Mettler-Toledo Halogen moisture balance (HB43S) measured the dry solids content to be 34.10%. Approximately 5 g of the industrial mash was added to 15 mL conical centrifuge tubes (Fisher). Each treatment was run in replicates of 4; all four treatments were run for 50 hours prior to HPLC analysis. Enzymes were dosed according to product specifications (Table 1) and the volume of stock solution to add to fermentation was found using the formula below.

$$Enz . dose ( ml ) = \frac{\begin{matrix}Final enz . dose ( mg EP / g DS ) \\ Mash weight ( g ) Solid content ( \% DS )\end{matrix}}{Conc . Enzyme ( mg EP / ml )}$$

Water was dosed into each sample such that the total added volume was equal across treatments.

TABLE 1
Enzyme dose

| | | | Enzyme |
|---|---|---|---|
| Enzyme | Dose | Units | Abbreviation |
| No Enzyme | — | — | Control |
| Glucoamylase Blend 10.5 | 30 | μg EP/g DS | GAB10.5 |
| Glucoamylase TC | 30 | μg EP/g DS | GATC |
| Glucoamylase DX | 30 | μg EP/g DS | GADX |
| Laminarinase AC | 30 | μg EP/g DS | LAC |
| Polygalacturonase UF | 30 | μg EP/g DS | PGUF |

Tubes were dosed with enzyme and incubated for 2 hours at 50° C. with vortexing every 15 minutes. After incubation, the tubes were allowed to cool before adding yeast to initiate fermentation. Rehydrated yeast (5.5 g Fermentis ETHANOL RED yeast in 100 mL 35° C. tap water incubated at 32° C. for 30 minutes) was dosed at 100 μl of yeast slurry per tube. Following the addition of yeast, the tubes were incubated at 32° C. in a water bath. Tubes were vortexed twice a day. After incubation, samples were stopped by the addition of 50 μl of 40% v/v H2SO4 and centrifuged at 1570×g (3000 rpm) for 10 minutes in a Beckman Coulture benchtop centrifuge (Allegra 6R) with rotor GH3.8 and then filtered into HPLC vials through 0.45 μm syringe filters (Whatman) into a 1.5 ml Eppendorf tube. Samples were centrifuged again in a Microfuge 18 (Beckman Coulture) at 18000×g (14000 rpm) for 10 minutes to remove more particulates. Samples were diluted 1:2 in mobile phase buffer (5 mM H2SO4) prior to submission for HPLC analysis.

### HPLC Analysis:

| | |
|---|---|
| HPLC system | Agilent's 1100/1200 series with Chem station software |
| | Degasser |
| | Quaternary Pump |
| | Auto-Sampler |
| | Column Compartment /w Heater |
| | Refractive Index Detector (RI) |
| Column | Bio-Rad HPX-87H Ion Exclusion Column 300 mm × |
| | 7.8 mm parts# 125-0140 |
| | Bio-Rad guard cartridge cation H parts# 125-0129, |
| | Holder parts# 125-0131 |
| Method | 0.005M H₂5O₄ mobile phase |
| | Flow rate of 0.6 ml/min |
| | Column temperature - 65° C. |
| | RI detector temperature - 55° C. |

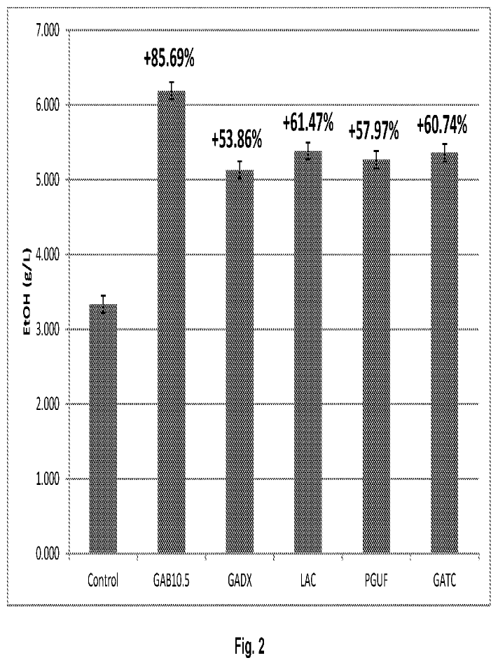

The method quantifies several analytes using calibration standards for dextrins (DP4+), maltotriose, maltose, glucose, fructose, acetic acid, lactic acid, glycerol and ethanol. A 4 point calibration including the origin is used.
The results of the ethanol fermentations are shown in FIG. 2.

1-23. (canceled)

24. A process of producing a fermentation product from starch containing material comprising:
(a) forming a slurry comprising the starch-containing material and water;
(b) converting the starch-containing material into dextrins with an alpha-amylase;
(c) saccharifying the dextrins using a carbohydrate source generating enzyme to form sugars;
(d) fermenting sugars using a fermenting organism;
(e) recovering the fermentation product to form whole stillage;
(f) separating the whole stillage into a liquid fraction thin stillage and solid fraction wet cake;
(g) hydrolyzing the thin stillage; and
(h) recycle a portion of the hydrolyzed thin stillage to steps (a);
wherein the thin stillage in step (g) is hydrolyzed using a glucoamylase and/or polygalactorunase.

25. The process of claim 24, wherein the thin stillage is hydrolyzed in step (g) with a glucoamylase (E.C. 3.2.1.3).

26. The process of claim 24, further comprising hydrolyzing the thin stillage in step (g) with a pullulanase (E.C. 3.2.1.41).

27. The process of claim 24, further comprising hydrolysing the thin stillage in step (g) with a laminarinase (E.C. 3.2.1.6).

28. The process of claim 24, wherein the thin stillage is hydrolysed in step (g) with a combination of glucoamylase and alpha-amylase.

29. The process of claim 24, wherein the thin stillage is hydrolysed in step (g) with a combination of glucoamylase and pullulanase.

30. The process of claim 24, wherein the thin stillage is hydrolysed in step (g) with a combination of polygalacturonase and laminarinase.

31. The process of claim 24, wherein alpha-amylase is added in step (a).

32. The process of claim 24, wherein a protease is added in step (a) or in step (b).

33. The process of claim 24, wherein a protease is added in step (a) and in step (b).

34. The process of claim 24, wherein a protease is present in step (a) or in step (b).

35. The process of claim 24, wherein steps (c) and (d) are carried out sequentially.

36. The process of claim 24, wherein steps (c) and (d) are carried out simultaneously.

37. The process of claim 24, wherein the portion of the hydrolyzed thin stillage that is not recycled as backset is evaporated to syrup and condensate.

38. The process of claim 37, wherein the condensate is recycled to step (a).

39. The process of claim 24, wherein between 5-90 vol-% of the hydrolyzed thin stillage is recycled as backset to step (a).

40. The process of claim 24, wherein the recycled hydrolyzed thin stillage constitutes from about 1-70 vol.-% of the slurry formed in step (a).

41. The process of claim 24, wherein the thin stillage is hydrolysed in step (g) at a temperature in the range from 20-80° C.

42. The process of claim 24, wherein the dry solids (DS) content in the thin stillage is in the range from 10-50% (W/W).

43. The process of claim 24, wherein the thin stillage is hydrolysed in step (g) for 0.1-10 hours.
